(19) United States
(12) Patent Application Publication
(10) Pub. No.: US 2002/0064837 A1
(43) Pub. Date: May 30, 2002

(54) METHOD FOR SYNTHESIZING A NUCLEIC ACID MOLECULE USING A RIBONUCLEASE

(76) Inventors: THUAN QUOC TRINH, GAITHERSBURG, MD (US); CHRISTIAN ELLIOTT GRUBER, FREDERICK, MD (US)

Correspondence Address: STERNE KESSLER GOLDSTEIN & FOX PLLC, ATTORNEYS AT LAW, 1100 NEW YORK AVENUE NW SUITE 600, WASHINGTON, DC, 200053934

(21) Appl. No.: 09/306,986

(22) Filed: May 7, 1999

Related U.S. Application Data
(60) Provisional application No. 60/084,737, filed on May 8, 1998.

(51) Int. Cl. C12Q 1/68

(52) U.S. Cl. 435/91.1; 435/6; 435/91.2

(57) ABSTRACT

The subject invention provides novel compositions containing one or more peptides or polypeptides which possess ribonuclease activity. Preferably the ribonucleases for inclusion in the compositions are ribonucleases that substantially lack deoxyribonuclease activity. A preferred embodiment is a composition comprising RNase A or RNase T1. Another aspect of the invention is to provide methods for synthesizing nucleic acids, typically DNA, using the compositions of the invention or one or more ribonucleases. Another aspect of the invention involves the use of the subject method of nucleic acid synthesis to carry out the synthesis step in a polymerase chain reaction experiment. Yet another aspect of the invention is to provide kits for the synthesis of nucleic acids, wherein the kits comprise one or more peptides or polypeptides that possess ribonuclease activity.

### INTRODUCTION

The applications of the polymerase chain reaction (PCR) technique, an in vitro enzymatic amplification of DNA, seem limitless. PCR has been used in methods including direct cloning from genomic DNA or cDNA, in vitro mutagenesis and engineering of DNA, analysis of allelic sequence variations, analysis of RNA transcript structure, genetic fingerprinting of forensic samples, assays for the presence of infectious agents, prenatal diagnosis of genetic diseases, genomic fingerprinting, and direct nucleotide sequencing of genomic DNA or cDNA, and others.

Typically, PCR involves the step of denaturing a polynucleotide, followed by the step of annealing at least a pair of primer oligonucleotides to the denatured polynucleotide, i.e., hybridizing the primer to the denatured polynucleotide template. After the annealing step, an enzyme with polymerase activity catalyzes synthesis of a new polynucleotide strand that incorporates the primer oligonucleotides and uses the original denatured polynucleotide as a synthesis template. PCR is described in numerous publications, including, PCR: A Practical Approach, M. J. McPherson, et al., IRL Press (1991), PCR Protocols: A guide to Methods and Applications, by Innis, et al. Academic Press (1990), and PCR technology: Principals and Applications for DNA Amplification, H. A. Britch, Stockton Press (1990).

The application of PCR to an increasing number of analyses has required the development of more efficient methods for acquiring samples to be analyzed. Therefore, methods for amplification of sequences from crude DNA have evolved as was shown for linear amplification DNA sequencing, for example, which can be done directly from crude preparation of DNA from bacteriophage plaques and bacterial colonies (Krishnan, B. R. et al. (1991) Nucl. Acids. Res. 19: 1153). However, these methods have not allowed consistent results in that in many instances, these methods have failed to produce the desired polynucleotide product. These failures may be attributable to a number of factors including such problems as template and primer base mismatches, and inefficient annealing of the primer to the template, among others. Therefore, there is a need for a method for optimizing nucleic acid synthesis, particularly by PCR, from crude DNA preparations.

### SUMMARY OF THE INVENTION

The present invention satisfies the need mentioned above.

Applicants have found that the problems associated with nucleic acid synthesis (particularly for PCR) from crude preparations may be due to the abundance of RNA in such crude DNA preparations. Consequently, the present invention provides compositions and methods for synthesizing polynucleotides in the presence of ribonucleases. These compositions and methods result in proper amplification and elongation of target DNA templates. Additionally, there is a significant increase in the amount of synthesized product, and increased product length.

Therefore, the subject invention provides novel compositions containing one or more enzymes, proteins or peptides (or fragments, mutants, derivatives or variants thereof) that possesses ribonuclease (RNase) activity. Preferably, such ribonucleases (or fragments, mutants, derivatives or variants thereof) substantially lack deoxyribonuclease (DNase) activity. More preferably, the ribonucleases and compositions used in the invention lack DNase activity (i.e. DNase-free). In another aspect, the ribonucleases used in the invention are thermostable and thus may be employed in high temperature nucleic acid synthesis reactions such as PCR. In this manner, the synthesis reaction may be conducted at elevated temperature without inactivating the ribonuclease activity. A preferred embodiment of the invention is a composition comprising DNase-free RNase A.

Another aspect of the invention provides a method for synthesizing nucleic acids, specifically DNA, using one or more enzymes, proteins or peptides (or fragments, mutants, derivatives or variants thereof) possessing RNase activity. Preferably, the ribonucleases used substantially lack DNase activity, and more preferably lack detectable levels of DNase activity. The method provided for synthesizing DNA (or other polynucleotides) comprises the step of mixing one or more desired templates with one or more enzyme, proteins or peptides (or fragments, mutants, derivatives or variants thereof) possessing RNase activity along with other reagents required for polynucleotide synthesis. Reagents required for polynucleotide synthesis include one or more nucleotides (e.g. dNTPs) or derivatives thereof, one or more polynucleotide primers, one or more DNA polymerases, and the like. The invention thus relates to a method of synthesizing a nucleic acid molecule comprising: (a) mixing a nucleic acid template with one or more DNA polymerases and with one or more RNases of the invention; and (b) incubating said mixture under conditions sufficient to synthesize a nucleic acid molecule complementary to all or a portion of said template. Thus, ribonuclease treatment may be conducted simultaneously with the nucleotide synthesis reaction and thus one or more ribonucleases may be added in conjunction other components necessary for a nucleotide synthesis (e.g. nucleotides, primers, one or more DNA polymerases and the like). In a related aspect, one or more ribonucleases may be added to a sample prior to the nucleic acid synthesis step. Thus, a sample may be treated in accordance with the invention with one or more ribonucleases and following such treatment, nucleic acid synthesis in the presence of one or more polymerases may be conducted. In this aspect, the ribonuclease activity may or may not be inactivated after treatment but before synthesis by well known techniques. Thus, ribonuclease treatment may be accomplished prior to and/or during the nucleic acid synthesis reaction.

Another aspect of the invention relates to amplification of nucleic acid molecules, for example a polymerase chain reaction or in an application of PCR, using one or more ribonucleases in accordance with the invention. The invention thus relates to a method for amplifying a double stranded DNA molecule, comprising: (a) providing a first and second primer, wherein said first primer is complementary to a sequence at or near the 3′-termini of the first strand of said DNA molecule and said second primer is complementary to a sequence at or near the 3′-termini of the second strand of said DNA molecule; (b) hybridizing said first primer to said first strand and said second primer to said second strand in the presence of one or more DNA polymerases and one or more RNases of the invention, under conditions such that a third DNA molecule complementary to said first strand and a fourth DNA molecule complementary to said second strand are synthesized; (c) denaturing said first and third strand, and said second and fourth strands; and (d) repeating steps (a) to (c) one or more times. For amplification of nucleic acid molecules, ribonuclease treatment may also be performed prior to and/or during nucleic acid synthesis or amplification. Thus, according to the invention, ribonucleases may be used at any step and may be removed or inactivated at any step. Removal or inactivation of ribonucleases can be accomplished using techniques well known to those in ordinary skill in the art (e.g. chemical extraction (phenol and/or chlorophorm), precipitation, protein denaturation, heat, etc.).

The invention also relates to conducting sequencing reactions in the presence of one or more ribonucleases of the invention. The invention thus relates to a method of sequencing a DNA molecule, comprising: (a) hybridizing a primer to a first DNA molecule; (b) contacting said molecule of step (a) with deoxyribonucleoside triphosphates, one or more DNA polymerases, one or more RNases of the invention, and one or more terminator nucleotides; (c) incubating the mixture of step (b) under conditions sufficient to synthesize a random population of DNA molecules complementary to said first DNA molecule, wherein said synthesized DNA molecules are shorter in length than said first DNA molecule and wherein said synthesized DNA molecules comprise a terminator nucleotide at their 3′ termini; and (d) separating said synthesized DNA molecules by size so that at least a part of the nucleotide sequence of said first DNA molecule can be determined. Such terminator nucleotides include ddTTP, ddATP, ddGTP, ddITP or ddCTP. As indicated above, treatment of samples with ribonucleases may take place prior to and/or during the sequencing reaction.

Another aspect of the invention provides kits for the synthesis, amplification, labeling or sequencing of nucleic acids, wherein the kits comprise one or more RNases of the invention. The kits may also contain other reagents useful in polynucleotide synthesis such as polynucleotide precursors, one or more nucleotides, one or more synthesis primers, one or more synthesis templates, one or more DNA polymerases, suitable buffers, and the like.

### BRIEF DESCRIPTION OF THE DRAWINGS

These and other features, aspects, and advantages of the present invention will become better understood with reference to the following description and appended claims, and accompanying drawings where:

FIG. 1 represents liquid culture colony PCR with and without RNase A. Sample colonies from a cDNA library which did not produce an amplification product when assayed using a PCR buffer without RNase were grown overnight in liquid media. 100 ul of cells were pelleted and resuspended in 100 ul of H₂O. PCR with and without RNase A was performed on 5 ul of cell culture. Amplification products were fractionated on a gel and stained with ethidium bromide (EtBr). Lane 1: 1 Kb ladder; lane 2: 100 bp ladder; lanes 3-14: samples 9, 10, 16, 25, 26, 28, 29, 30, 37, 42, 43 and 44 assayed by PCR without RNase; lane 15: 100 bp ladder; lanes 16-28, samples 9, 10, 16, 25, 26, 28, 29, 30, 37, 42, 43 and 44 assayed by PCR with RNase; lane 29: 100 bp ladder; lane 30: 1 Kbp ladder.

FIG. 2 represents plated colony PCR with and without RNase A. As described for FIG. 1, sample colonies from a cDNA library which did not produce an amplification product when assayed using a PCR buffer without RNase were grown overnight in liquid media. 5 ul of each of the fresh cultures were dotted onto an amplicillin plate and grown overnight. PCR with and without RNase A was performed on 0.5 ul of bacteria from a plated colony transferred directly to tubes subjected to PCR cycling without prior preparation of cell lysates. Amplification products were fractionated on a gel and stained with ethidium bromide (EtBr). Lane 1: 1 kb ladder; lane 2: 100 bp ladder; lanes 3-14: samples 9, 10, 16, 25, 26, 28, 29, 30, 37, 42, 43 and 44 assayed by PCR without RNase; lane 15: 100 bp ladder; lanes 16-28: samples 9, 10, 16, 25, 26, 28, 29, 30, 37, 42, 43 and 44 assayed by PCR with RNase; lane 29: 100 bp ladder; lane 30: 1 Kbp ladder.

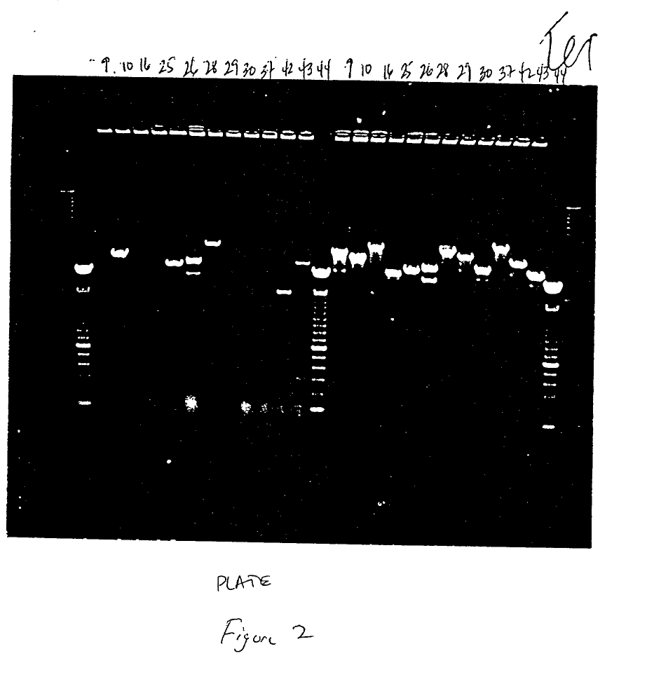

### DETAILED DESCRIPTION

### Definitions

In the description that follows, a number of terms used in recombinant DNA technology are extensively utilized. In order to provide a clearer and consistent understanding of the specification and claims, including the scope to be given such terms, the following definitions are provided.

Hybridization.

The terms “hybridization” and “hybridizing” refers to the pairing of two complementary single-stranded nucleic acid molecules (RNA and/or DNA) to give a double-stranded molecule. As used herein, two nucleic acid molecules may be hybridized, although the base pairing is not completely complementary. Accordingly, mismatched bases do not prevent hybridization of two nucleic acid molecules provided that appropriate conditions, well known in the art, are used.

Template.

The term “template” as used herein refers to a double-stranded or single-stranded nucleic acid molecule (RNA or DNA or messenger RNA) which is to be amplified, synthesized or sequenced. In the case of a double-stranded DNA molecule, denaturation of its strands to form a first and a second strand is performed before these molecules may be amplified, synthesized or sequenced. A primer, complementary to a portion of a template is hybridized under appropriate conditions and one or more DNA polymerases or other polymerases may then synthesize a DNA molecule complementary to said template or a portion thereof. The newly synthesized molecule, according to the invention, may be equal or shorter in length than the original template. Mismatch incorporation during the synthesis or extension of the newly synthesized molecule may result in one or a number of mismatched base pairs. Thus, the synthesized molecule need not be exactly complementary to the template.

Amplification.

As used herein “amplification” refers to any in vitro method for increasing the number of copies of a nucleotide sequence with the use of one or more DNA polymerases. Nucleic acid amplification results in the incorporation of nucleotides into a DNA molecule or primer thereby forming a new DNA molecule complementary to all or a portion of the template. The formed DNA molecule and its template can be used as templates to synthesize additional DNA molecules. As used herein, one amplification reaction may consist of many rounds of DNA replication. DNA amplification reactions include, for example, polymerase chain reactions (PCR). One PCR reaction may consist of 20 to 100 “cycles” of denaturation and synthesis of a DNA molecule.

Cloning vector.

A plasmid, cosmid or phage DNA or other DNA molecule which is able to replicate autonomously in a host cell, and which is characterized by one or a small number of restriction endonuclease recognition sites at which such DNA sequences may be cut in a determinable fashion without loss of an essential biological function of the vector, and into which DNA may be spliced in order to bring about its replication and cloning. The cloning vector may further contain a marker suitable for use in the identification of cells transformed with the cloning vector. Markers, for example, are tetracycline resistance or ampicillin resistance.

Expression vector.

A vector similar to a cloning vector but which is capable of enhancing the expression of a gene which has been cloned into it, after transformation into a host. The cloned gene is usually placed under the control (i.e., operably linked to) certain control sequences such as promoter sequences.

Recombinant host.

Any prokaryotic or eukaryotic or microorganism which contains the desired cloned genes in a expression vector, cloning vector or any DNA molecule. The term “recombinant host” is also meant to include those host cells which have been genetically engineered to contain the desired gene on the host chromosome or genome.

Incorporating.

The term “incorporating” as used herein means becoming a part of a DNA molecule or primer.

Nucleic acid.

“Nucleic acid” refers to a synthetic or natural molecule comprising a covalently linked sequence of nucleotides which are joined by a phosphodiester bond between the 3′ position of the pentose of one nucleotide and the 5′ position of the pentose of the adjacent nucleotide.

Substantially pure.

As used herein “substantially pure” means that the desired purified protein is essentially free from contaminating cellular contaminants which are associated with the desired protein in nature. Contaminating cellular components may include, but are not limited to, phosphatases, exonucleases, endonucleases or undesirable DNA polymerase enzymes.

Primer.

As used herein “primer” refers to a single-stranded oligonucleotide that is extended by covalent bonding of nucleotide monomers during amplification or polymerization of a DNA molecule.

Nucleotide.

As used herein “nucleotide” refers to a base-sugar-phosphate combination. Nucleotides are monomeric units of a nucleic acid sequence (DNA and RNA). The term nucleotide includes deoxyribonucleoside triphosphates such as dATP, dCTP, dITP, dUTP, dGTP, dTTP, or derivatives thereof. Such derivatives include, for example, [αS]dATP, 7-deaza-dGTP and [αS]dTTP, [αS]dGTP, [αS]dCTP, 7-deaza-dATP. The term nucleotide as used herein also refers to dideoxyribonucleoside triphosphates (ddNTPs) and their derivatives. Illustrated examples of dideoxyribonucleoside triphosphates include, but are not limited to, ddATP, ddCTP, ddGTP, ddITP, ddTTP. According to the present invention, a “nucleotide” may be unlabeled or detectably labeled by well known techniques. Detectable labels, include, for example, radioactive isotopes, fluorescent labels, chemiluminescent labels, bioluminscent labels and enzyme labels.

Ribonuclease.

As used herein “ribonuclease” (RNase) refers to a peptide, polypeptide or protein (or mutant, fragment, derivative or variant thereof) which is able to cleave or digest or degrade RNA. Ribonucleases of the present invention can be purified from an organism, can be synthetically produced, or can be produced by recombinant techniques by cloning one or more genes involved in production of an RNase and isolating the RNase from the recombinant host cell. The activity of a ribonuclease can be detected by measuring hydrolysis of RNA into acid-soluble material. Ribonucleases can be nonspecific endoribonucleases meaning that they are able to hydrolyze RNA nonspecifically as opposed to specific endoribonucleases which can cleave RNA at a specific sequence. Specific and nonspecific endoribonucleases are useful in the present invention. Ribonucleases for use in the invention may also be exoribonucleases. The ribonucleases for use in the present invention are preferably substantially lacking deoxyribonuclease (DNase) activity. The term “substantially lacking” when used with respect to DNase activity, refers to RNase which completely lacks or which contains an amount of DNase which will not substantially degrade the DNA contained in a sample during a nucleic acid synthesis reaction. Preferably, RNases used in the invention lack DNase or contain an amount of DNase activity which does not substantially degrade or does not degrade the DNA template during a nucleic acid synthesis reaction such as PCR. RNases can be made substantially lacking DNase activity by boiling the RNase sample (typically at a concentration of 10 mg/ml in H₂O) for about 10-30 minutes, a method well known in the art. Ribonucleases with different sequence specificities are known. Depending on the enzyme and buffer conditions, the RNA digested can be single stranded RNA, double stranded RNA, or part of a RNA/DNA duplex, or all three. The buffer conditions for each RNase are specified by their respective suppliers and are commonly known in the art.

Prior to the inventors' work, polynucleotide synthesis in vitro was performed without RNase. In a variety of nucleic acid synthesis procedures, the subject compositions provide superior synthesis results, as compared with synthesis results obtained without RNase. The composition is especially useful in DNA synthesis when the sample is crude, i.e. prepared rapidly such that it contains contaminating RNA. In such situations, the results achieved, i.e., the amount of synthesis product produced, are significantly greater than the amount of synthesis product obtained without RNase. Other advantages of the subject compositions and methods include increased product length, as well as the synthesis of polynucleotides that could not be synthesized previously, i.e., in the absence of RNase.

The subject invention thus provides novel compositions for use in synthesizing nucleic acids, particularly DNA. The subject compositions comprise one or more ribonucleases and may optimally further comprises one or more DNA polymerases. Such composition may also comprise one or more components selected from the group consisting of one or more nucleotides, one or more primers, one or more buffers suitable for nucleic acid synthesis and/or one or more templates.

Enzymes, protein or peptide (or fragment, mutant, variant or derivatives thereof) possessing RNase activity for use in the present compositions and methods may be isolated from natural sources, produced through recombinant DNA techniques, or chemically synthesized. Such enzymes that possess RNase activity and their properties are detailed in The Enzymes, Vol. IV (P. D. Boyer ed.) Academic Press, San Diego. Examples of enzymes that possess RNase activity useful in the compositions and methods of the present invention include RNase A, RNase H, RNase T1, RNase T2, RNase S, RNase B, RNase C or variants, derivatives, fragments or mutants thereof and the like.

RNase A, a preferred enzyme for use in the present invention, is an endoribonuclease from bovine pancreas that hydrolyzes RNA after C (cytosine) and U (Uracil) residues [Richard and Wyckoff (1971) In The Enzymes, Vol. IV (P. D. Boyer, ed.) pp.647-806. Academic Press, San Diego]. Cleavage occurs between the 3′-phosphate group of a pyrimidine ribonucleotide and the 5′-hydroxyl of the adjacent nucleotide. The reaction generates a 2′:3′ cyclic phosphate which then is hydrolyzed to the corresponding 3′-nucleoside phosphates.

Ribonuclease T1 from Aspergillus oryzae is an endoribonuclease that hydrolyzes RNA after G residues [Uchida and Egami (1971) In: The Enzymes, Vol IV (P. D. Boyer, Ed.) pp. 205-250. Academic Press, San Diego]. Cleavage occurs between the 3′-phosphate group of a guanine ribonucleotide and the 5′-hydroxyl of the adjacent nucleotide. The reaction generates a 2′:3′ cyclic phosphate which then is hydrolyzed to the corresponding 3′-nucleoside phosphates.

RNase A and RNase T1 are extremely difficult to inactivate, and are active under a wide range of reaction conditions, and thereby naturally thermostable. RNase A and RNase T1 cleave single stranded and double-stranded RNA as well as the RNA strand in RNA:DNA duplexes at low salt concentrations (0-100 mM NaCl). However, at NaCl concentrations of 0.3 M or above, RNase A and RNase T1 become specific for cleavage of single-stranded RNA. Removal of RNase A or RNase T1 from a reaction solution generally requires treatment with proteinase K followed by multiple phenol extractions and ethanol precipitation.

In a specific embodiment, when adding RNase A, it is preferable that the salt concentration of the mixture be optimized for the digestions desired. As described above, at low salt concentrations, below about 100 mM NaCl, RNase digests the RNA strand of RNA:DNA duplexes as well as single stranded and double stranded RNA. At salt concentrations above 100 mM NaCl, RNase digests single and double stranded RNA, but not the RNA strand of RNA:DNA duplexes.

RNase T2 also from Aspergillus oryzae is a nonspecific endoribonuclease that hydrolyzes RNA at the 3′-phosphate group of an adenosine residue.

RNase H from E. coli is an endoribonuclease that specifically hydrolyzes the phosphodiester bonds of RNA in RNA:DNA duplexes to generate products with 3′ hydroxyl and 5′ phosphate ends [Berkower et al. (1973) J. Biol. Chem. 248:5914-5921].

Other RNases useful in the present invention include RNase B, RNase C, and RNase S available for example from Sigma, St. Louis, Mo.

In addition, one or more RNases (e.g. two or more, three or more, four or more etc.) may be combined to carry out the methods of the invention or may be combined in the composition or kits of the invention. The number of ribonucleases may range from 2 to 5, 2 to 4 and 2 to 3. Thus, specific ribonucleases can be added alone or in combination such that the interfering RNA is hydrolyzed. Some of these specific RNases are listed in Table 1.

TABLE 1
RNases with sequence specificity.

| RNase | Sequence Specificity |
|---|---|
| U2 | Ap↓N |
| CL3 | C(A/G)p↓N |

Other RNases for use in the invention may also be used and will be readily identified by one of ordinary skill in the art. Such RNases are preferably substantially lacking, in DNase activity.

When using the subject compositions in reaction mixtures that are exposed to elevated temperature, e.g., during the PCR technique, use of thermostable RNase is preferred. The term “thermostable” when used with respect to an enzyme, is readily understood by a person of ordinary skill in the art. Typically, a “thermostable” enzyme retains at least 50 percent of its activity after exposure to a temperature of 80° C. for a period of 20 minutes.

In accordance with the invention, the amount of RNases and the conditions used may be determined by one skilled in the art using the assays described herein. Typically, the concentration of RNase(s), the incubation time and temperature and the order of addition may vary depending on the RNase used, the amount of RNA in the sample, and the desired result. Preferably, the RNase(s) are added prior to beginning the nucleic acid synthesis reaction or during the synthesis reaction at a final concentration ranging between about 2 ug/ml to about 5 mg/ml depending on the abundance of RNA in the sample, preferably about 20 μg/ml to about 400 μg/ml, still more preferably about 50 μg/ml to about 300 μg/ml, and most preferably about 200 μg/ml, for RNase A, and about 0.5-500 Units of RNase T1 per microliter of a reaction mixture, preferably about 1 to 200 Units, more preferably about 2 to 50 Units and most preferably 20 Units of RNase T1/ul of a reaction mixture, where a Unit of RNase T1 is defined as the amount of enzyme required to hydrolyze 1 A₂₆₀ unit of yeast RNA to acid-soluble material in 15 minutes at 37° C.

A variety of polypeptides having polymerase activity are useful in accordance with the present invention. Included among these polypeptides are enzymes such as nucleic acid polymerases (including DNA polymerases). Such polymerases include, but are not limited to, Thermus thermophilus (Tth) DNA polymerase, Thermus aquaticus (Taq) DNA polymerase, Thermotoga neopolitana (Tne) DNA polymerase, Thermotoga maritima (Tma) DNA polymerase, Thermococcus litoralis (Tli or VENT™) DNA polymerase, Pyrococcus furiosus (Pfu) DNA polymerase, DEEPVENT™ DNA polymerase, Pyrococcus woosii (Pwo) DNA polymerase, Bacillus sterothermophilus (Bst) DNA polymerase, Bacillus caldophilus (Bca) DNA polymerase, Sulfolobus acidocaldarius (Sac) DNA polymerase, Thermoplasma acidophilum (Tac) DNA polymerase, Thermus flavus (Tfl/Tub) DNA polymerase, Thermus ruber (Tru) DNA polymerase, Thermus brockianus (DYNAZYME™) DNA polymerase, Methanobacterium thermoautotrophicum (Mth) DNA polymerase, mycobacterium DNA polymerase (Mtb, Mlep), and mutants and variants and derivatives thereof.

Polymerases used in accordance with the invention may be any enzyme that can synthesize a nucleic acid molecule from a nucleic acid template, typically in the 5′ to 3′ direction. The nucleic acid polymerases used in the present invention may be mesophilic or thermophilic, and are preferably thermophilic. Preferred mesophilic DNA polymerases include T7 DNA polymerase, T5 DNA polymerase, Klenow fragment DNA polymerase, DNA polymerase III and the like. Preferred thermostable DNA polymerases that may be used in the methods of the invention include Taq, Tne, Tma, Pfu, Tfl, Tth, Stoffel fragment, VENT™ and DEEPVENT™ DNA polymerases, and mutants, variants and derivatives thereof (U.S. Pat. No. 5,436,149; U.S. Pat. No. 4,889,818; U.S. Pat. No. 4,965,188; U.S. Pat. No. 5,079,352; U.S. Pat. No. 5,614,365; U.S. Pat. No. 5,374,553; U.S. Pat. No. 5,270,179; U.S. Pat. No. 5,047,342; U.S. Pat. No. 5,512,462; WO 92/06188; WO 92/06200; WO 96/10640; Barnes, W. M., Gene 112:29-35 (1992); Lawyer, F. C., et al., PCR Meth. Appl. 2:275-287 (1993); Flaman, J.-M, et al., Nucl. Acids Res. 22(15):3259-3260 (1994)). For amplification of long nucleic acid molecules (e.g., nucleic acid molecules longer than about 3-5 Kb in length), at least two DNA polymerases (one substantially lacking 3′ exonuclease activity and the other having 3′ exonuclease activity) are typically used. See U.S. Pat. No. 5,436,149; U.S. Pat. No. 5,512,462; Barnes, W. M., Gene 112:29-35 (1992), the disclosures of which are incorporated herein in their entireties. Examples of DNA polymerases substantially lacking in 3′ exonuclease activity include, but are not limited to, Taq, Tne(exo⁻), Tma(exo⁻), Pfu(exo⁻), Pwo(exo⁻) and Tth DNA polymerases, and mutants, variants and derivatives thereof.

Polypeptides having nucleic acid polymerase activity are preferably used in the present methods at a final concentration in solution of about 0.1-200 Units per milliliter, about 0.1-50 Units per milliliter, about 0.1-40 Units per milliliter, about 0.1-3.6 Units per milliliter, about 0.1-34 Units per milliliter, about 0.1-32 Units per milliliter, about 0.1-30 Units per milliliter, or about 0.1-20 Units per milliliter, and most preferably at a concentration of about 20 Units per milliliter. Of course, other suitable concentrations of nucleic acid polymerases suitable for use in the invention will be apparent to one of ordinary skill in the art.

Other components in a nucleic acid synthesis reaction may include one or more components selected from the group consisting of one or more synthesis primers, one or more synthesis templates, one or more polynucleotide precursors for incorporation into the newly synthesized polynucleotide, (e.g. dATP, dCTP, dGTP, dTTP), and the like. Detailed methods for carrying out polynucleotide synthesis are well known to the person of ordinary skill in the art and can be found, for example, in Molecular Cloning, second edition, Sambrook et al., Cold Spring Harbor Laboratory Press, Cold Spring Harbor, N.Y. (1989). The process of PCR employs a polynucleotide synthesis step in each cycle; this polynucleotide synthesis step may be achieved using the subject invention.

Methods envisioned in accordance with the invention include nucleic acid synthesis, sequencing, labeling and amplification from crude preparations of DNA from any cell or tissue such as viral, bacteriophage, bacteria, insect, bird, fish, plant, yeast, prokaryotes, eukaryotes and mammals. Preparation of crude extracts of cells or tissues may be accomplished by standard procedures which allows removal of at least some nucleic acids from the cell or tissue without the need for purification of the nucleic acids from the cells, tissue or cell/tissue debri, although nucleic acids may be isolated or purified or partially purified prior to use in accordance with the invention. Examples of such procedures includes lysis or disruption of cells or tissues by mechanical (heat, sonication, vortex with glass beads, etc.), enzymatic (lysozyme, etc.) or chemical (pH, salt, detergent, etc.) means. Alternatively, the cells and/or tissues may be used directly in the methods of the invention without prior manipulation (such as lysis, disruption etc.). The applications of the invention are numerous, including direct cloning from genomic DNA or cDNA, in vitro mutagenesis and engineering of DNA, analysis of allelic sequence variations, analysis of RNA transcript structure, genetic fingerprinting of forensic samples, autopsies, biopsies, and archeological samples, assays for the presence of infectious agents, prenatal diagnosis of genetic diseases, genomic fingerprinting, and direct nucleotide sequencing of genomic DNA or cDNA, to name a few.

As is well known, sequencing reactions (isothermal DNA sequencing and cycle sequencing of DNA) require the use of polymerases. Dideoxy-mediated sequencing involves the use of a chain-termination technique which uses a specific polymer for extension by DNA polymerase, a base-specific chain terminator and the use of polyacrylamide gels to separate the newly synthesized chain terminated DNA molecules by size so that at least a part of the nucleotide sequence of the original DNA molecule can be determined. Specifically, a DNA molecule is sequenced by using four separate DNA sequence reactions, each of which contains different base-specific terminators (or one reaction if fluorescent terminators are used). For example, the first reaction will contain a G-specific terminator, the second reaction will contain a T-specific terminator, the third reaction will contain an A-specific terminator, and a fourth reaction may contain a C-specific terminator. Preferred terminator nucleotides include dideoxyribonucleoside triphosphates (ddNTPs) such as ddATP, ddTTP, ddGTP, ddITP and ddCTP. Analogs of dideoxyribonucleoside triphosphates may also be used and are well known in the art.

When sequencing a DNA molecule, ddNTPs lack a hydroxyl residue at the 3′ position of the deoxyribose base and thus, although they can be incorporated by DNA polymerases into the growing DNA chain, the absence of the 3′-hydroxy residue prevents formation of the next phosphodiester bond resulting in termination of extention of the DNA molecule. Thus, when a small amount of one ddNTP is included in a sequencing reaction mixture, there is competition between extension of the chain and base-specific termination resulting in a population of synthesized DNA molecules which are shorter in length than the DNA template to be sequenced. By using four different ddNTPs in four separate enzymatic reactions, populations of the synthesized DNA molecules can be separated by size so that at least a part of the nucleotide sequence of the original DNA molecule can be determined. DNA sequencing by dideoxy-nucleotides is well known and is described by Sambrook et al., In: Molecular Cloning, a Laboratory Manual, Cold Spring Harbor Laboratory Press, Cold Spring Harbor, N.Y. (1989). As will be readily recognized, the polymerases of the present invention may be used in such sequencing reactions.

As is well known, detectably labeled nucleotides are typically included in sequencing reactions. Any number of labeled nucleotides can be used in sequencing (or labeling) reactions, including, but not limited to, radioactive isotopes, fluorescent labels, chemiluminescent labels, bioluminescent labels, and enzyme labels. For example, the polymerases of the present invention may be useful for incorporating αS nucleotides ([αS]dATP, [αS]dTTP, [αS]dCTP and [αS]dGTP) during sequencing (or labeling) reactions.

Polymerase chain reaction (PCR), a well known DNA amplification technique, is a process by which DNA polymerase and deoxyribonucleoside triphosphates are used to amplify a target DNA template. In such PCR reactions, two primers, one complementary to the 3′ termini (or near the 3′-termini) of the first strand of the DNA molecule to be amplified, and a second primer complementary to the 3′ termini (or near the 3′-termini) of the second strand of the DNA molecule to be amplified, are hybridized to their respective DNA strands. After hybridization, DNA polymerase, in the presence of deoxyribonucleoside triphosphates, allows the synthesis of a third DNA molecule complementary to the first strand and a fourth DNA molecule complementary to the second strand of the DNA molecules. Such double stranded DNA molecules may then be used as DNA templates for synthesis of additional DNA molecules by providing a DNA polymerase, primers and deoxyribonucleoside triphosphates. As is well known, the additional synthesis is carried out by “cycling” the original reaction (with excess primers and deoxyribonucleoside triphosphates) allowing multiple denaturing and synthesis steps. Typically, denaturing of double stranded DNA molecules to form single stranded DNA templates is accomplished by high temperatures. The DNA polymerases are preferably heat stable DNA polymerases, and thus will survive such thermal cycling during DNA amplification reactions.

The invention also relates to amplification or synthesis cDNA. As is known, cDNA is prepared from mRNA templates. See U.S. Pat. Nos. 5,405,776 and 5,244,797. The double stranded cDNA is typically cloned into a host cell and such host cells may be used in the present invention.

The invention herein also contemplates a kit format which comprises a package unit having one or more containers comprising one or more RNases of the invention and in some embodiments including containers of various reagents used for polynucleotide synthesis, including synthesis in PCR, sequencing, amplification, and labeling of nucleic acid molecules by well known techniques, depending on the content of the kit. Such kits may comprise a carrying means being compartmentalized to receive in close confinement one or more container means such as vials, test tubes, and the like. Each of such container means comprises components or a mixture of components needed to perform nucleic acid synthesis, sequencing, labeling, or amplification, reactions.

A kit used for amplifying or synthesis of nucleic acids will comprise, for example, a first container means comprising a ribonuclease or combination of ribonucleases of the invention and one or a number of additional container means which comprise a single type of nucleotide or mixtures of nucleotides. The kit may also contain one or more of the following items: polymerization enzymes, primers, buffers, instructions, and controls. Kits may include containers of reagents mixed together in suitable proportions for performing the methods in accordance with the invention. Reagent containers preferably contain reagents in unit quantities that obviate measuring steps when performing the subject methods.

A kit for sequencing may comprise a number of container means. A first container means may, for example, comprise one or more RNases of the invention. A second container means may comprise a polymerase of combination of polymerases. A third container may comprise one or a number of types of nucleotides needed to synthesize a DNA molecule complementary to a DNA template. A fourth container means may comprise one or more or a number of different types of terminators (such as dideoxynucleoside triphosphates). A fifth container means may comprise pyrophosphatase. In addition to the above container means, additional container means may be included in the kit which comprise one or a number of primers and/or a suitable sequencing buffer.

When desired, the kit of the present invention may also include container means which comprise detectably labeled nucleotides which may be used during the synthesis or sequencing of a nucleic acid molecule. One of a number of labels may be used to detect such nucleotides. Illustrative labels include, but are not limited to, radioactive isotopes, fluorescent labels, chemiluminescent labels, and bioluminescent labels and enzymes.

The following examples are provided to illustrate the present invention, and should not be construed as limiting thereof.

### EXAMPLE 1

### Single Colony PCR

Samples from a cDNA library which could not be amplified using a reaction buffer without RNase were grown in 1 ml of LB (100 ug amplicillin/ml) overnight at 30° C. Also, 5 ul from each of the fresh cultures were dotted on an ampicillin plate and grown overnight at 30° C.

The next day, PCR reactions were prepared from bacteria grown in liquid media and plated bacteria.

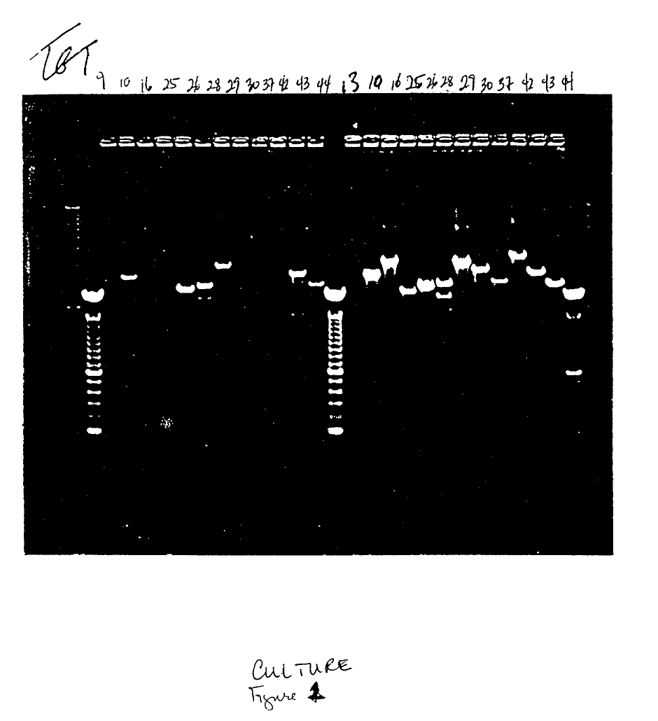

One hundred microliters from each liquid culture were pelleted and resuspended in 100 ul H₂O. PCR with and without RNase was performed on 5 ul of cell culture (FIG. 1). The cells were resuspended in 20 ul of colony PCR buffer with or without RNase A.

Colony PCR buffer contained 2 mM MgSO₄, 18 mM (NH₄)SO₄, 60 mM Tris-SO₄, 200 nM each dNTP, 500 nM each primer, 0.5 ul of eLONGase™ (Life Technologies, Gaithersburg, Md.) mix and H₂O to 25 ul. For PCR with RNase A, RNase A was added to the PCR buffer at 200 ug/ml. RNase A was dissolved in water at 10 mg/ml and boiled for 15 minutes prior to use.

Similarly, about 0.5 ul of E. coli cells from a plated colony were picked with a pipette tip and transferred to 0.2 ml tubes for PCR cycling without any prior preparation of cell lysate. The colonies were resuspended in 25 ul of colony PCR buffer with and without RNase A.

PCR cycling conditions were as follows:

| | | |
|---|---|---|
| Pre-amplification cell lysis and denaturation | 94° C. | 60 s |
| Thermal cycling: | | |
| Denaturation | 94° C. | 15-30 s |
| Annealing | 50° C. | 20-30 s |
| Extension | 68° C. or 72° C. | 5 min. |
| Cycling was repeated 34 times. | | |

A fraction of the cycling reaction after PCR assay was removed and fractionated on a gel and stained with EtBr to visualize DNA bands.

Results show that addition of RNase into the PCR buffer solution resulted in a superior amplification reaction, where there was a higher product yield and the product was of a higher molecular weight, indicating complete amplification of the template without interruptions of the DNA polymerase. Similar results were achieved when RNase T1 at a concentration of 500 Units/reaction was added instead of RNase A.

### Conclusions

1. Without RNase, some colonies did not allow any amplification at all. Others would yield shorter, inaccurate DNA amplification bands.

2. Those PCR products which failed in reaction buffer without using RNase, were typically large cDNA inserts which were amplified in a reaction buffer containing RNase.

3. Using RNase significantly eliminates the RNA background which obscures the PCR products at 100 to 300 bp.

### EXAMPLE 2

### Single Colony Sequencing

Cells from 100 ul of fresh overnight culture of DH10B cells containing pRPA-1 plasmid grown in LB at 100 ug ampicillin/ml were pelleted. These cells were then resuspended in one of the following solutions:

25 ul of 100 ug/ml RNase A

25 ul of 40 ug/ml proteinase K

25 ul of water

25 ul of 100 ug/ml RNase A; incubate at 37° C. for 15 min followed by addition of 5 ul of 200 ug/ml proteinase K.

Each sample was then incubated at 37° C. for 15 min followed by a second incubation at 80° C. for 15 min. Debris was removed by centrifugation for five minutes. The supernatant was then used in ³²P-end-labeled primer cycle sequencing using Taq DNA polymerase using the dsDNA Cycle Sequencing System (Life Technologies, Inc., Rockville, Md.). See also Craxton (1991) Methods: A Companion to Methods in Enzymology 3,20. Purified pRPA-1 DNA was used as control along with pUC19 (Life Technologies, Inc.). All reactions were run in duplicate.

The program used for cycle sequencing was:

| | |
|---|---|
| 20 cycles: | 95° C. × 30 s |
| | 55° C. × 30 s |
| | 70° C. × 60 s |
| followed by 10 cycles: | 95° C. × 30 s |
| | 70° C. × 60 s |

An improvement in the sequence quality was found as compared with the samples without RNase A digestion indicating the usefulness of adding RNase A during a sequencing reaction.

### EQUIVALENTS

All publications and patent applications mentioned in this specification are indicative of the level of skill of those skilled in the art to which this invention pertains. All publications and patent applications are herein incorporated by reference to the same extent as if each individual publication or patent application was specifically and individually indicated to be incorporated by reference.

The foregoing written specification is considered to be sufficient to enable one skilled in the art to practice the invention. Indeed, various modifications of the above-described modes for carrying out the invention which are obvious to those skilled in the field of molecular biology or related fields are intended to be within the scope of the following claims.

What is claimed is:

1. A kit for synthesizing a nucleic acid molecule, said kit comprising a peptide or polypeptide having ribonuclease activity.

2. A kit according to claim 1, wherein said peptide or polypeptide is selected from the group consisting of RNase A, RNase T1, RNase H, RNase S, RNase B, RNase C, and RNase T2 or fragments, variants, derivatives or mutants thereof.

3. A kit according to claim 1, wherein said peptide or polypeptide is thermostable.

4. A kit according to claim 1, further comprising one or more components selected from the group consisting of:
a) one or more nucleotides;
b) one or more DNA polymerases;
c) one or more suitable buffers for nucleic acid synthesis; and
d) one or more primers.

5. A kit according to claim 4, wherein said DNA polymerase is thermostable.

6. A kit according to claim 5, wherein said thermostable DNA polymerase is selected from the group consisting of Taq DNA polymerase, Tne DNA polymerase, Tma DNA polymerase, Tth DNA polymerase, Tli or VENT™ DNA polymerase, Pfu DNA polymerase, DEEPVENT™ DNA polymerase, Pwo DNA polymerase, Bst DNA polymerase, Bca DNA polymerase, and Tfl DNA polymerase or fragments, variants, derivatives or mutants thereof.

7. A kit according to claim 4, wherein one or more of said nucleotides are detectably labeled.

8. A method for synthesizing a nucleic acid molecule, said method comprising:
a) mixing a nucleic acid template, with one or more DNA polymerases, and one more peptides or polypeptides having ribonuclease activity; and
b) incubating said mixture under condition sufficient to synthesize a nucleic acid molecule complementary to all or a portion of said template.

9. The method according to claim 8, wherein said peptide or polypeptide having ribonuclease activity is selected from the group consisting of RNase A, RNase T1, RNase H, RNase S, RNase B, RNase C, and RNase T2 or fragments, variants, derivatives or mutants thereof.

10. The method according to claim 8, said mixture further comprising one or more components selected from the group consisting of:
a) one or more nucleotides;
b) one or more DNA polymerases;
c) one or more suitable buffers for nucleic acid synthesis; and
d) one or more primers.

11. The method according to claim 8, wherein said DNA polymerase is thermostable.

12. The method according to claim 11, wherein said thermostable DNA polymerase is selected from the group consisting of Taq DNA polymerase, Tne DNA polymerase, Tma DNA polymerase, Tth DNA polymerase, Tli or VENT™ DNA polymerase, Pfu DNA polymerase, DEEPVENT™ DNA polymerase, Pwo DNA polymerase, Bst DNA polymerase, Bca DNA polymerase, and Tfl DNA polymerase or fragments, variants, derivatives or mutants thereof.

13. The method according to claim 10, wherein one or more of said nucleotides are detectably labeled.

14. A composition for synthesizing a nucleic acid molecule, said composition comprising one or more peptides or polypeptides having ribonuclease activity.

15. The composition according to claim 14, wherein said peptide or polypeptide is selected from the group consisting of RNase A, RNase T1, RNase H, RNase S, RNase B, RNase C, and RNase T2 or fragments, variants, derivatives or mutants thereof.

16. The composition according to claim 14, wherein said polypeptide or peptide is thermostable.

17. The composition according to claim 14, further comprising one or more components selected from the group consisting of:
a) one or more nucleotides;
b) one or more DNA polymerases;
c) one or more suitable buffers for nucleic acid synthesis;
d) one or more primers; and
e) one or more templates.

18. The composition according to claim 17, wherein said DNA polymerase is thermostable.

19. The composition according to claim 18, wherein said thermostable DNA polymerase is selected from the group consisting of Taq DNA polymerase, Tne DNA polymerase, Tma DNA polymerase, Tth DNA polymerase, Tli or VENT™ DNA polymerase, Pfu DNA polymerase, DEEPVENT™ DNA polymerase, Pwo DNA polymerase, Bst DNA polymerase, Bca DNA polymerase, and Tfl DNA polymerase or fragments, variants, derivatives or mutants thereof.

20. The composition according to claim 14, wherein said composition further comprises one or more DNA polymerases.

21. A method of sequencing a DNA molecule, comprising:
a) mixing a first DNA molecule with one or more polymerases, and one or more peptides or polypeptides having ribonuclease activity;
b) hybridizing a primer to said first DNA molecule;
c) contacting said DNA molecule of step (b) with deoxyribonucleoside triphosphates, and one or more terminator nucleotides;
d) incubating the mixture of step (c) under conditions sufficient to synthesize a random population of DNA molecules complementary to said first DNA molecule, wherein said synthesized DNA molecules are shorter in length than said first DNA molecule and wherein said synthesized DNA molecules comprise a terminator nucleotide at their 5′ termini; and
e) separating said synthesized DNA molecules by size so that at least a part of the nucleotide sequence of said first DNA molecule can be determined.

22. The method according to claim 21, wherein said peptide or polypeptide is selected from the group consisting of RNase A, RNase T1, RNase H, RNase S, RNase B, RNase C, and RNase T2 or fragments, variants, derivatives or mutants thereof.

23. The method according to claim 21, wherein said peptide or polypeptide is thermostable.

24. The method according to claim 21, wherein said deoxyribonucleoside triphosphates are selected from the group consisting of dATP, dCTP, dGTP, dTTP, dITP, 7-deaza-dGTP, dUTP, [α-S]dATP, [α-S]dTTP, [α-S]dGTP, and [α-S]dCTP.

25. The method according to claim 21, wherein said terminator nucleotides are selected from the group consisting of ddATP, ddCTP ddGTP, ddITP, ddTTP.

26. The method according to claim 21, wherein one or more of said deoxyribonucleoside triphosphates is detectably labeled.

27. The method according to claim 21, wherein one or more of said terminator nucleotides is detectably labeled.

28. A kit for sequencing a DNA molecule comprising one or more peptides or polypeptides having ribonuclease activity.

29. The kit according to claim 28, wherein said peptide or polypeptide is selected from the group consisting of RNase A, RNase T1, RNase H, RNase S, RNase B, RNase C, and RNase T2 or fragments, variants, derivatives or mutants thereof.

30. The kit of claim 28, further comprising one or more components selected from the group consisting of
a) one or more dideoxyribonucleoside triphosphates
b) one or more deoxyribonucleoside triphosphates;
c) one or more DNA polymerases;
d) one or more suitable buffers for nucleic acid synthesis; and
e) one or more primers.

31. A method for amplifying a double stranded DNA molecule, comprising:
a) providing a first and second primer, wherein said first primer is complementary to a sequence at or near the 3′ termini of the first strand of said DNA molecule and said second primer is complementary to a sequence at or near the 3′-termini of the second strand of said DNA molecule;
b) hybridizing said first primer to said first strand and said second primer to said second strand in the presence of one or more peptides or polypeptides having ribonuclease activity and one or more DNA polymerases under conditions such that a third DNA molecule complementary to said first strand and a fourth DNA molecule complementary to said second strand are synthesized;
c) denaturing said first and third strand, and said second and fourth strands; and
d) repeating steps (a) to (c) one or ore times.

32. The method of claim 31, wherein said peptide or polypeptide is selected from the group consisting of RNase A, RNase T1, RNase H, RNase S, RNase B, RNase C, and RNase T2 or fragments, variants, derivatives or mutants thereof.

33. The method according to claim 31, wherein said peptide or polypeptide is thermostable.

34. The method according to claim 31, wherein said DNA polymerase is thermostable.

35. The method according to claim 34, wherein said thermostable DNA polymerase is selected from the group consisting of Taq DNA polymerase, Tne DNA polymerase, Tma DNA polymerase, Tth DNA polymerase, Tli or VENT™ DNA polymerase, Pfu DNA polymerase, DEEPVENT™ DNA polymerase, Pwo DNA polymerase, Bst DNA polymerase, Bca DNA polymerase, and Tfl DNA polymerase or fragment, variants, derivatives or mutants thereof.

36. A kit for amplifying a nucleic acid molecule comprising one or more peptides or polypeptides having ribonuclease activity.

37. The kit according to claim 36 further comprising one or more components selected from the group consisting of:
a) one or more nucleotides;
b) one or more DNA polymerases;
c) one or more suitable buffers for nucleic acid synthesis; and
d) one or more primers.
